(19) United States
(12) Patent Application Publication
(10) Pub. No.: US 2024/0149038 A1
(43) Pub. Date: May 9, 2024

(54) CONNECTION SYSTEM FOR MEDICAL APPLICATIONS

(71) Applicant: B. Braun Melsungen AG, Melsungen (DE)

(72) Inventor: Ralf Kunschak, Willisau (CH)

(21) Appl. No.: 18/567,176

(22) PCT Filed: Jun. 8, 2022
(86) PCT No.: PCT/EP2022/065464

§ 371 Date: Dec. 5, 2023

(30) Foreign Application Priority Data
Jun. 8, 2021 (DE) 10 2021 205 771.3

(51) Int. Cl. A61M 39/10

(52) U.S. Cl. CPC A61M 39/1011

(57) ABSTRACT

A connecting system for medical applications, which can be used in medical technology, particularly in the field of oncology, includes a male connector and a complementary female connector. The male connector has a plug and an annular wall that concentrically encloses the plug while forming an annular gap. The male connector also has an internal thread facing toward the plug. The female connector has a socket complementary to the plug and an external thread complementary to the internal thread. The female connector is inserted into the annular gap of the male connector in a connected state. An inactive adhesive arrangement is provided in the annular gap and assigned an activation system, in such a way that activation of the adhesive arrangement takes place as a result of insertion of the female connector into the annular gap of the male connector.

### CROSS-REFERENCE TO RELATED APPLICATIONS

This application is the United States national stage entry of International Application No. PCT/EP2022/065464, filed on Jun. 8, 2022, and claims priority to German Application No. 10 2021 205 771.3, filed on Jun. 8, 2021. The contents of International Application No. PCT/EP2022/065464 and German Application No. 10 2021 205 771.3 are incorporated by reference herein in their entireties.

### FIELD

The invention relates to a connecting system for medical applications, having a male connector and having a complementary female connector, wherein the male connector has a plug as well as an annular wall, which concentrically encloses the plug while forming an annular gap and has an internal thread facing toward the plug, and wherein the female connector has a socket complementary to the plug as well as an external thread complementary to the internal thread and in the connected state is inserted into the annular gap of the male connector.

### BACKGROUND

A Luer lock connecting system is widely known from medical technology. Luer lock connecting systems are used to connect medical fluid systems or to attach medical syringes to catheters or other medical attachment systems. Such Luer lock connecting systems are used for the releasable connection of corresponding medical functional sections. In some medical application cases, it is necessary that a connection between two medical functional parts can no longer be released once it is closed.

For instance, it is known from US 2006/0047251 A1 for a Luer lock connection to be subsequently fixed by introducing a liquid adhesive and therefore made nonreleasable (FIG. 4 of US 2006/0047251 A1 and associated description).

### SUMMARY

It is an object of the invention to provide a connecting system of the type mentioned in the introduction, which allows reliable use in medical specialist fields in which there is a high health risk for patients and users.

This object is achieved in that an inactive adhesive arrangement is provided in the annular gap and is assigned an activation system, in such a way that activation of the adhesive arrangement takes place as a result of insertion of the female connector into the annular gap of the male connector. By the solution according to the invention, an adhesive arrangement is positioned permanently in the annular gap of the male connector but is activated only when connection to the female connector is carried out. The activation of the adhesive arrangement in this case takes place automatically with the connecting process between the female Luer lock connector and the male connector, without additional method steps subsequently being necessary. Subsequent introduction of adhesive after the connection has been carried out, as in the prior art, is advantageously avoided by the solution according to the invention. Medical specialist fields with a high health risk for patients and users are found particularly in oncology. The activation system may be assigned indirectly or directly to the adhesive arrangement, as will be explained in more detail below. Indirect assignment is preferably intended to mean mechanical encapsulation of the adhesive arrangement. Direct assignment is intended to mean physical properties or chemical properties of the adhesive arrangement, which allow activation of the adhesive arrangement only under particular physical or chemical conditions. The adhesive arrangement may be configured with one component or several components. Corresponding adhesive components of the adhesive arrangement may be separated from one another in the inactive state. Corresponding adhesive components include not only components of a two-component adhesive, but also primers which are used to pretreat a material to be bonded, or fragrances which are perceptible to a user upon release. Because the adhesive arrangement is received in the annular gap in the inactive state, handling of the connecting system overall and of the male connector in particular may be carried out in the same way as for connecting systems which are not provided with an adhesive arrangement. Corresponding users therefore do not need to be specially trained in order to be able to use the connecting system according to the invention. The activation of the adhesive arrangement necessarily takes place in a simple way by the connecting of the female connector to the male connector, without additional method steps needing to be carried out or particular guidelines having to be observed by the user. The connecting system according to the invention for medical applications includes all types of medical connecting systems which comprise a male connector having a plug, which is especially in the form of a tip and which in particular is conical or cylindrical, and having an annular wall which concentrically encloses the plug while forming an annular gap and has an internal thread facing toward the plug, as well as a female connector having a socket complementary to the plug and an external thread complementary to the internal thread. The annular wall of the male connector may in this case be configured integrally with the plug or as a separately produced component, which is connected to the plug. The male connector and the female connector preferably have standardized geometries, such as are known in particular from ISO 80369-7:2021 (E), DIN EN ISO 80369-3 of December 2016 or DIN EN ISO 80369-6 of October 2018. The connecting system according to the invention may be used particularly advantageously for a Luer lock connecting system having male and female connectors that are provided with standardized geometries according to the aforementioned ISO 80369-7:2021 (E).

In one configuration of the invention, the activation system comprises encapsulation of the adhesive arrangement by means of at least one capsule. Advantageously, the adhesive arrangement comprises at least one liquid, flowable or paste-like adhesive component, which after the destruction of the capsule necessarily flows or is displaced between corresponding wall surfaces of the thread turns of the female and male connectors and is cured. The at least one capsule encloses a corresponding adhesive component of the adhesive arrangement tightly. The encapsulation may be provided by a single-layer or multilayer envelope of the adhesive components, consisting of the same or different capsule materials. A corresponding capsule may therefore also be configured with one or several layers. One or more capsules may be provided for each adhesive component. The capsules are used to enclose primers in the same way as to enclose components of a two-component adhesive or to enclose a single-component adhesive.

In another configuration of the invention, outer dimensions of the at least one capsule filled with the adhesive arrangement are matched to inner dimensions of the annular gap in such a way that the capsule can be received in the annular gap. Preferably, corresponding capsules with an integrated adhesive component or with an integrated primer have diameters of up to 2 mm. In the encapsulated state of the adhesive arrangement, the capsules have a three-dimensional shape of a closed hollow body, in particular a spherical or ovoid shape or another geometrical hollow body shape.

In another configuration of the invention, the adhesive arrangement comprises different adhesive components, which are accommodated in different capsules. In the case of a two-component adhesive, the two different adhesive components are separated from one another by the capsules so long as the male and female connectors are not connected to one another. A primer, which renders a nonadherent material of the male connector or of the female connector adherent before the actual adhesive of the connecting system according to the invention starts to act, is also envisioned as an adhesive component.

In another configuration of the invention, the capsules with the different adhesive components are positioned in the annular gap in accordance with an activation sequence of the adhesive components. The capsules are positioned in such a way that, when the female connector is inserted into the annular gap of the male connector, the capsules with the adhesive components which are necessary for an active connection to the next adhesive components or to the material of the connectors are initially destroyed. When a primer is used to pretreat corresponding surfaces of the annular gap of the male connector or of the complementary connecting faces of the female connector, the at least one capsule filled with the primer is destroyed first when the female connector penetrates, in order to be able to flow along the corresponding surfaces. During the further penetration of the female connector, the capsules which contain the adhesive components for the nonreleasable connection between the female connector and the male connector are thereupon destroyed. All the adhesive components of the adhesive arrangement, i.e. including a corresponding primer, are liquid or flowable in their state when encapsulated in the respective capsule, in order to be able to penetrate reliably after the destruction of the corresponding capsule into corresponding cavities between the internal thread of the annular gap of the male connector and the external thread of the female connector.

In another configuration of the invention, the at least one capsule is configured with such a low resistance that destruction of the capsule with release of the at least one adhesive component takes place during connection of the female connector to the male connector by contact between the female connector and the capsule. The destruction is preferably carried out by shear and/or compressive stresses on the capsule due to the female connector being screwed in. This ensures that release of the adhesive arrangement necessarily also takes place with the connection between the female and male connectors, so that fixing of the connection between the female and male connectors is ensured. The fixing is obtained when the at least one adhesive component is cured.

In another configuration of the invention, the capsule is configured in such a way that destruction of the capsule when the female connector is connected to the male connector can be perceived by a user by means of a sense organ of the user. The destruction of the capsule is perceptible by the user in particular haptically, acoustically or in another way, for example by the release of incorporated fragrances. This configuration allows a user to check that the fixing of the male and female connectors relative to one another is ensured. Suitable fragrances may be added to an adhesive component. Alternatively, it is possible to accommodate corresponding fragrances in their own capsules.

In another configuration of the invention, the at least one capsule provided with an adhesive component is fixed in the annular gap with a force or form fit or materially. This ensures that the corresponding hollow body consisting of a capsule and adhesive component does not accidentally fall out of the annular gap of the male connector when the male connector is being handled. Force-fit or form-fit fixing is preferably carried out by corresponding dimensioning of the at least one capsule and consequently of the correspondingly configured hollow body, so that the capsule including the adhesive component is introduced into the annular gap with corresponding deformation, without being damaged or destroyed. Material fixing of the hollow body and therefore of the capsule, including the adhesive component enclosed by the latter, may be carried out by an additional adhesive or by a solvent, which partially dissolves a surface of the connector and thus allows fixing of the capsule on a corresponding wall section of the annular gap of the male connector. Alternatively or in addition, it is possible to provide a solvent which does not attack the surface of the connector but attacks a surface of a capsule material of the capsule, and thus allows the desired fixing of the capsule in the annular gap upon contact of the capsule with a corresponding wall section of the annular gap of the male connector.

In another configuration of the invention, the activation system has a chemical or physical activation function, which is assigned to the adhesive arrangement. Such an adhesive arrangement may be introduced without encapsulation directly into the annular gap of the male connector. The adhesive arrangement may be combined with an encapsulated primer, which allows chemical activation and then curing of the adhesive arrangement. The unencapsulated adhesive arrangement remains in the flowable state until the connection between the male connector and the female connector is established. The curing then takes place. For all adhesive components of the adhesive arrangements described, the activation is intended to mean the possibility of converting the corresponding adhesive component from a liquid, flowable or paste-like state into a cured state. In the case of an unencapsulated adhesive arrangement in the annular gap of the male connector, it is also possible to provide corresponding connecting faces of the female connector, which are inserted into the annular gap, with a substance that reacts with the adhesive arrangement contained in the annular gap so that the adhesive arrangement contained in the annular gap is activated by the contact with this substance.

In another configuration of the invention, the male connector and/or the female connector has a visually or haptically recognizable coding, which allows a user to distinguish it from a known connecting system without an adhesive arrangement. The coding is geometrically configured in a suitable way. Preferably, the connecting system according to the invention is color-marked in order allow a user to distinguish it from other connecting systems. This ensures that the user does not accidentally use a connection with which permanent fixing of the male and female connectors relative to one another cannot be carried out.

### BRIEF DESCRIPTION OF THE DRAWINGS

Further advantages and features of the invention may be found in the following description of preferred exemplary embodiments of the invention, which are represented with the aid of the drawings.

FIG. 1 schematically shows an embodiment of a connecting system according to the invention in the form of a Luer lock connecting system;

FIG. 2 shows an enlarged sectional representation of a male Luer lock connector of the Luer lock connecting system according to FIG. 1;

FIG. 3 shows a further male Luer lock connector according to a further embodiment of a Luer lock connecting system according to the invention;

FIG. 4 shows a sectional representation of a male Luer lock connector of a further Luer lock connecting system according to one embodiment of the invention;

FIG. 5 shows a sectional representation of a male Luer lock connector according to a further embodiment of a Luer lock connecting system according to the invention;

FIG. 6a shows a very enlarged sectional representation of the bearing of a capsule filled with an adhesive component on a wall section of an annular gap of the male Luer lock connector;

FIG. 6b shows an enlarged representation of a detail of FIG. 6a;

FIG. 7a shows a similar representation to FIG. 6a;

FIG. 7b shows an enlarged representation of the detail according to FIG. 7a;

FIG. 8a shows a further sectional representation similar to FIGS. 6a and 7a;

FIG. 8b shows an enlarged detail according to FIG. 8a;

FIG. 9 shows a male Luer lock connector according to a further embodiment of a Luer lock connecting system according to the invention;

FIG. 10 shows a male Luer lock connector according to a further embodiment of a Luer lock connecting system according to the invention;

FIG. 11 shows a partial representation of the Luer lock connecting system in the connected state between a female Luer lock connector and the Luer lock connector according to FIG. 10;

FIG. 12 shows an enlarged representation of a detail of the arrangement according to FIG. 11; and

FIG. 13 shows a male Luer lock connector according to a further embodiment of a Luer lock connecting system according to the invention.

### DETAILED DESCRIPTION

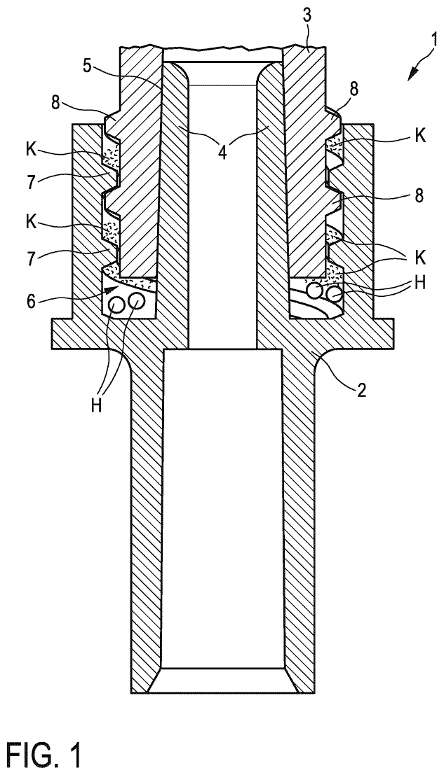

A medical connecting system in the form of a Luer lock connecting system 1 according to FIG. 1 comprises a male Luer lock connector 2 and a female Luer lock connector 3 (only partially represented). The male Luer lock connector 2 is provided in the widely known way with a Luer plug 4, onto which a Luer socket 5 is fitted in the connected state. The Luer plug 4 of the male Luer lock connector 2 and the Luer socket 5 of the female Luer lock connector 3 are conically configured complementarily to one another. The male Luer lock connector 2 furthermore has an annular wall, which concentrically encloses the Luer plug 4 at a radial distance and is separated from an outer lateral wall of the Luer plug 4 by an annular gap 6. The annular wall has an internal thread 7 protruding radially into the annular gap, which is provided with one or more thread turns. On its outer circumference, the female Luer lock connector 3 has an external thread 8 complementary to the internal thread 7, which in a similar way to the internal thread 7 is provided with one or more thread turns. Both the female Luer lock connector 3 and the male Luer lock connector 2 are respectively produced in one piece from a plastic material.

For a tight plug connection, the female Luer lock connector 3 with its Luer socket 5 is fitted onto an upper part of the Luer plug 4 of the male Luer lock connector 2, a lower extreme edge of an annular lateral surface of the Luer socket 5 of the female Luer lock connector 3 already protruding partially into the annular gap 6 of the male Luer lock connector 2. By subsequent rotation about a concentric longitudinal midaxis between the male Luer lock connector 2 and the female Luer lock connector 3, the thread turns of the external thread 8 and of the internal thread 7 necessarily engage in one another so that the socket 5 of the female Luer lock connector 3 and the Luer plug 4 of the male Luer lock connector 2 are drawn axially against one another until a tight connection is achieved between the outer lateral surface of the Luer plug 4 and an inner lateral surface of the Luer socket 5.

In some application cases, it is advantageous for a permanent and nonreleasable connection to be established between the male Luer lock connector 2 and the female Luer lock connector 3. The solution according to the invention is suitable for this. Various embodiments of Luer lock connecting systems 1 will be described below with the aid of FIGS. 1 to 13, which in addition to the male Luer lock connector 2 and the female Luer lock connector 3 provide an adhesive arrangement K that is provided inactively in the region of the male Luer lock connector 2 and is activated only when the female Luer lock connector 3 is screwed into the annular gap 6 of the male Luer lock connector 2. Activation is intended to mean activation of the adhesive properties of the adhesive arrangement K, which leads to curing of the adhesive arrangement K and consequently to fixing of the screwed connecting location between the male Luer lock connector 2 and the female Luer lock connector 3. The embodiments of Luer lock connecting systems 1 according to the invention, which are described below and represented with the aid of FIGS. 1 to 13, are always based on identical male and female Luer lock connectors, which are respectively provided with the same reference signs but with the addition of correspondingly consecutive lower-case letters to distinguish the various embodiments. The Luer lock connecting systems 1 represented and described differ merely in respect of corresponding adhesive arrangements and corresponding activation systems for these adhesive arrangements.

A feature common to all the embodiments according to FIGS. 1 to 13 is that both the adhesive arrangement and a correspondingly associated activation system are always accommodated in the region of the annular gap 6 to 6f of the respective male Luer lock connector 2. The respective adhesive arrangement K and an associated activation system are thereby accommodated inside the annular gap so that they are protected.

According to one embodiment (not represented) of a Luer lock connecting system 1 according to the invention, the male Luer lock connector 2 and/or the female Luer lock connector 3 are provided with a color coding, which is provided on the male Luer lock connector 2 and/or on the female Luer lock connector in such a way that a user can visually recognize that the corresponding Luer lock connecting system 1 is provided with an adhesive arrangement and an associated activation system. This makes it easier to distinguish it from known Luer lock connecting systems with which permanent fixing of the connection between the female Luer lock connector 3 and the male Luer lock connector 2 is not enabled. Color coding may be carried out by coloring at least one of the two connectors directly during production or by subsequent application of color markings.

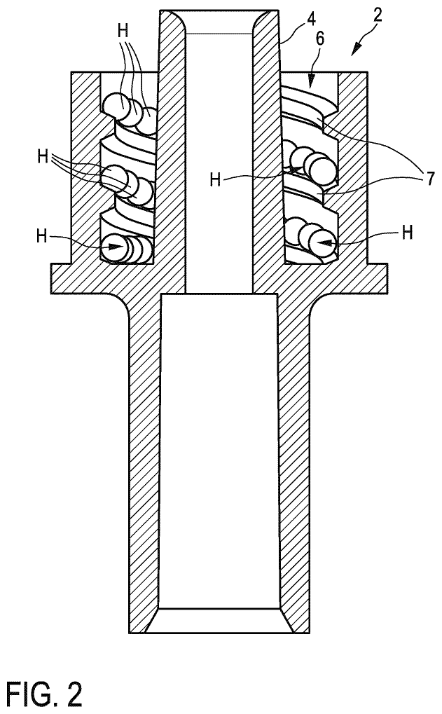

In the Luer lock connecting system 1 according to FIGS. 1 and 2, an adhesive arrangement K is provided by introducing a multiplicity of small capsules H, which are respectively filled with a liquid or flowable adhesive component, in the annular gap 6 of the male Luer lock connector 2. Each capsule H is configured substantially spherically and forms a closed hollow body that encloses the adhesive component. As may be seen with the aid of FIG. 2, in the Luer lock connecting system 1 according to FIGS. 1 and 2 the spherical capsules H are introduced along the threading of the internal thread 7 of the annular gap 6, so that the capsules H are arranged distributed over the entire length of the threading of the internal thread 7. Corresponding outer dimensions of the capsules H are designed in such a way that the capsules H in the filled state can be introduced into the annular gap 6 from above and are supported respectively on an upper flank of the threading of the internal thread 7. The capsules H are made from a capsule material which is destroyed under compressive or shear stresses that occur when the male Luer lock connector 2 and the female Luer lock connector 3 are screwed together, and releases the adhesive component. However, the capsule material is configured to be resistant enough that it is not damaged by machine or manual handling during the introduction into the annular gap 6. The capsules H with the enclosed adhesive components provide information to the user in the event of destruction during the screwing between the male and female Luer lock connectors 2, 3, either by acoustically perceptible bursting or tearing of the capsule material, by an initially increased and subsequently reduced resistance during the screwing, or by the release of incorporated auxiliary substances, for example fragrances.

As soon as the capsule material of the capsules H is destroyed, the adhesive component emerges and flows according to the representation of FIG. 1 into corresponding gap-like cavities between an outer contour of the female Luer lock connector 3 and an inner contour of the annular gap 6 of the male Luer lock connector 2. When the female Luer lock connector 3 is screwed in, the adhesive arrangement K is distributed at least substantially uniformly along the thread turns of the internal thread 7 and of the external thread 8. FIG. 1 shows a state of the female Luer lock connector 3 when partially screwed in. In the state fully screwed in, the capsules H which are not yet destroyed in FIG. 1 are also destroyed so that the adhesive arrangement K is also distributed between a base of the annular gap 6 and a lower extreme edge of the Luer socket 5 of the female Luer lock connector 3. The adhesive arrangement K is selected in such a way that relatively rapid curing takes place, preferably from within seconds up to 10 minutes. In the cured state, the adhesive arrangement K is so rigid that significant relative movement between the female Luer lock component 3 and the male Luer lock component 2 is no longer possible in the axial direction or in the screwing direction.

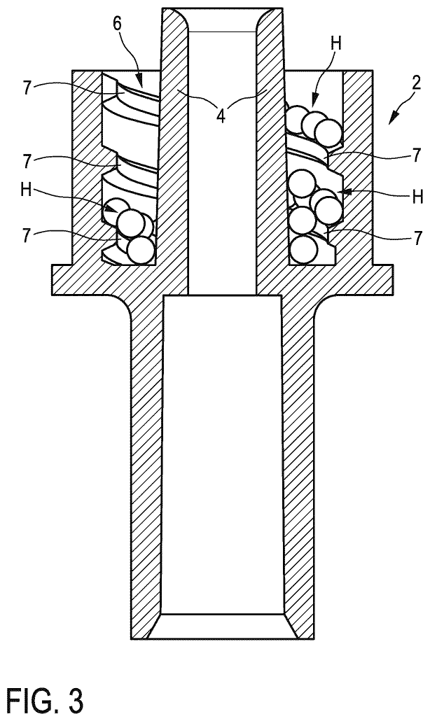

In the embodiment according to FIG. 3, the capsules H are not distributed entirely uniformly over the length of the threading of the internal thread 7, but instead are arranged concentratedly in the region of a base of the annular gap 6. In other regards, the comments already made above relating to the Luer lock connecting system 1 apply for this embodiment.

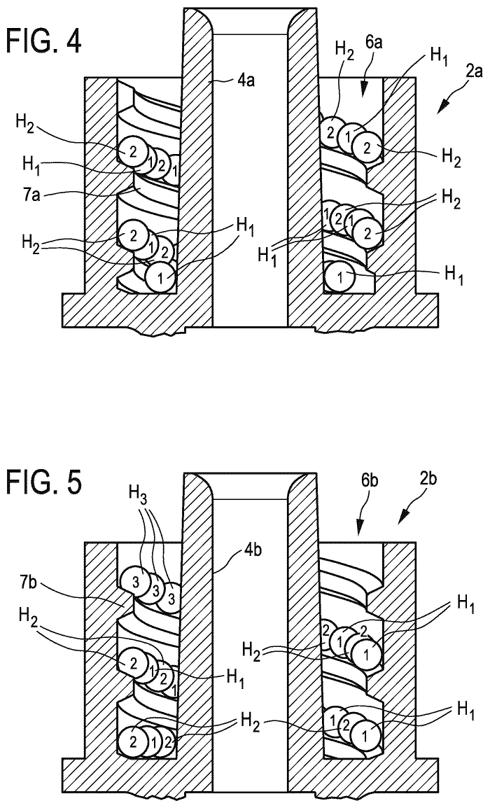

The embodiment according to FIG. 4 differs only in that the adhesive arrangement is provided in the form of a two-component adhesive, one of the two adhesive components respectively being accommodated in its own capsule H₁, H₂. A corresponding number of capsules H₁ and H₂ are provided according to the specific mixing ratio which is required for the two-component adhesive in order to achieve sufficient curing. Furthermore, the capsules H₁ and H₂ are arranged uniformly distributed in the threading of the annular gap 6a in such a way that, when the female Luer lock connector is screwed into the male Luer lock connector 2a, uniform mixing of the two adhesive components always takes place from the very start to the end of the screwing process. In the embodiment according to FIG. 4, a mixing ratio of 1:1 is provided. Correspondingly, equal numbers of capsules H₁ and capsules H₂ are then provided and are positioned uniformly distributed in the annular gap 6a.

A two-component adhesive is likewise used in the embodiment according to FIG. 5. Correspondingly, equal numbers of capsules H₁ and capsules H₂ with the different adhesive components are arranged distributed in the annular gap 6b of the male Luer lock connector 2b. The difference in this embodiment according to FIG. 5 is that in addition to the adhesive arrangement, a primer which is used to activate corresponding surfaces of the annular gap 6b or of the associated female Luer lock connector is also assigned. The use of such a primer is envisioned when the female and/or male Luer lock connector 2b consists of a material that forms no adhesive bond, or at most forms a weak adhesive bond, with the adhesive arrangement provided. Corresponding surfaces of the male and female Luer lock connectors are activated by the primer so that improved adhesion of the adhesive arrangement may take place on the respective surface. The primer is also enclosed in liquid or flowable form by a capsule material while forming capsules H₃, as may be seen in FIG. 5. Since activation of the corresponding surfaces is initially necessary for good material connection between the male and female Luer lock connectors, the capsules H₃ filled with the primer are arranged at the very top in the annular gap 6b so that the capsules H₃ are destroyed first, and the primer is released, when the female Luer lock connector is screwed onto the male Luer lock connector 2b. During the further screwing, the capsules H₁ and H₂ are destroyed in a similar way to the embodiments described above, so that the adhesive components of the adhesive arrangement are mixed.

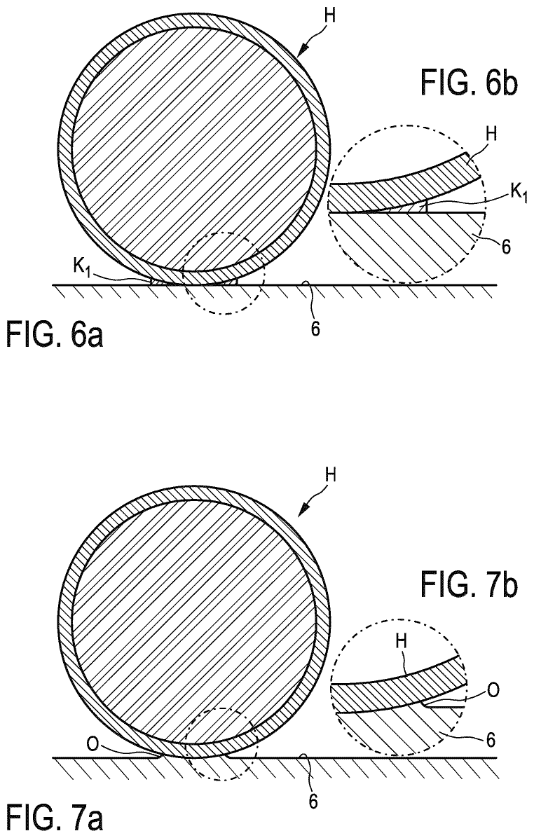

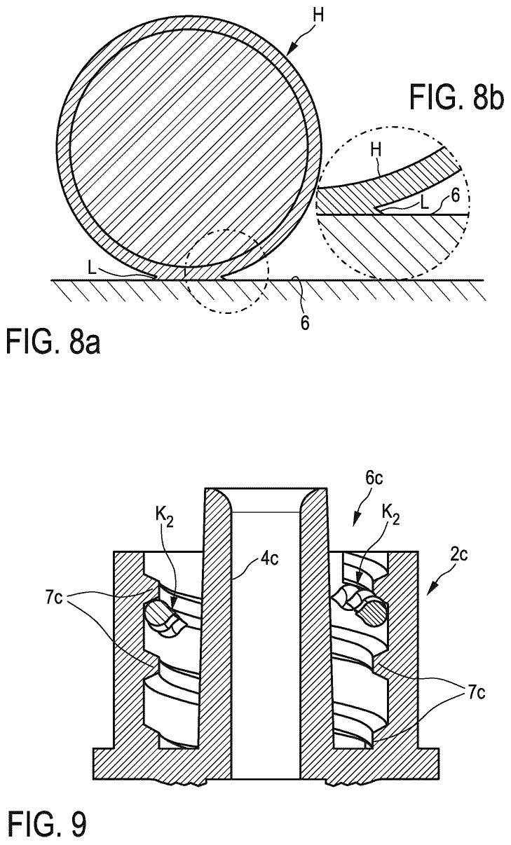

FIGS. 6a to 8b show various embodiments by means of which the capsules H in the annular gap 6 of the respective male Luer lock connector 2 can be fixed while they are being kept in the ready state of the male Luer lock connector, and therefore in the inactive state of the adhesive arrangement, before they are destroyed. In the embodiment according to FIGS. 6a and 6b, this is done by fixing the capsules H on the surface of the annular gap 6 by means of an additional adhesive K₁. In the embodiment according to FIGS. 7a and 7b, a solvent O which leads to partial dissolving of the corresponding surfaces of the annular gap 6 is used instead of an adhesive K₁, as a result of which the partially dissolved region O forms a connection to the capsule material of the respective capsule H. In the embodiment according to FIGS. 8a and 8b, a different solvent L is used to partially dissolve the capsule material of the respective capsule H itself, this solvent L advantageously likewise being applied in the region of the corresponding surfaces of the annular gap 6. The partial dissolving of the capsule material of the respective capsule H leads similarly to a connection between the surface of the annular gap 6 and the capsule material of the capsule H.

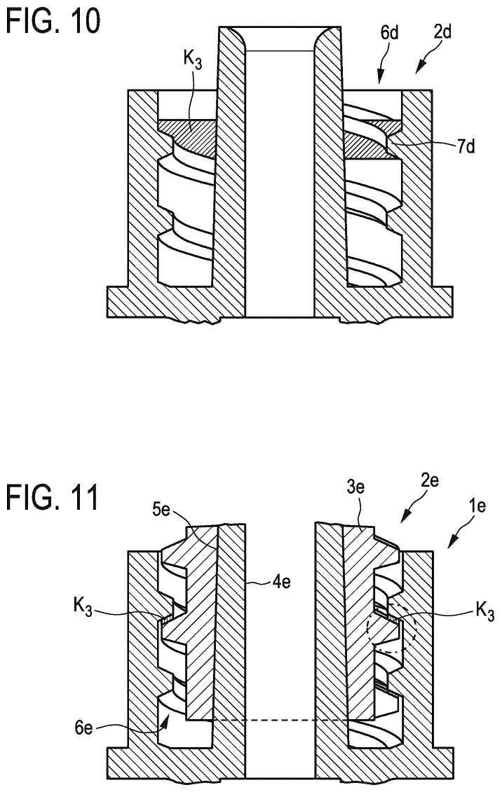

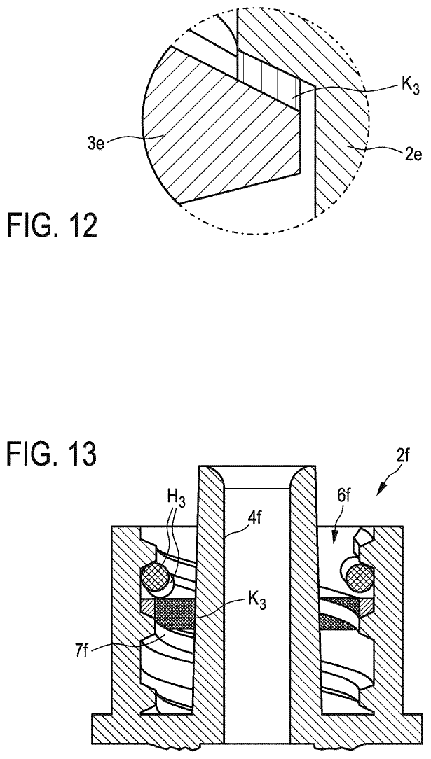

In the embodiment according to FIG. 9, an adhesive arrangement K₂ is provided in the form of a multiplicity of adhesive drops, without there being an additional envelope by means of a capsule material. Instead of in the form of drops, the unencapsulated adhesive arrangement K₂ may also be introduced into the annular gap 6c in any other desired portioning forms. The adhesive arrangement K₂ may preferably be formed by a hot-melt adhesive. Instead of a multiplicity of small portions of the adhesive arrangement K₂, according to FIG. 10 a corresponding adhesive arrangement K₃ may also be introduced in the form of a ring just below an entry region of the annular gap 6d in the annular gap of the male Luer lock connector 2d. The adhesive arrangement K₂ or K₃ is preferably configured in the form of a paste, in order on the one hand to allow consistent positioning in the ready state inside the annular gap 6c, 6d, and on the other hand to ensure good distribution of the adhesive arrangement K₂, K₃ when screwing in a female Luer lock connector. FIG. 11 represents a state of a female Luer lock connector 3e when screwed onto the Luer plug 4e of the male Luer lock connector 2e, in which the adhesive arrangement K₃ in the ready state has been positioned inside the annular gap 6e in the way which is shown with the aid of FIG. 10 for the male Luer lock connector 2d. The enlarged detail according to FIG. 12 shows the material connection by the adhesive arrangement K₃ between complementary flanks of the thread turns of the male Luer lock connector 2e and of the female Luer lock connector 3e.

In the embodiment according to FIG. 13, as was described with the aid of FIGS. 10 to 12, the adhesive arrangement K₃ is also preceded by a primer which is positioned in the form of the capsules H₃ above the adhesive arrangement K₃ in the ready state of the male Luer lock connector 2f When screwing in the female Luer lock connector, the primer is therefore released before the adhesive arrangement K₃ is distributed in the annular gap 6f by screwing in the female Luer lock connector, so that the corresponding contact faces of the female Luer lock connector and of the male Luer lock connector 2f have already been activated by the primer before the adhesive arrangement K₃ is distributed and activated, and subsequently cured.

1. A connecting system for medical applications, the connecting system comprising:
a male connector;
a female connector; and
an adhesive arrangement that is inactive,
the male connector comprising a plug and an annular wall that concentrically encloses the plug while forming an annular gap,
the male connector also comprising an internal thread facing toward the plug,
the female connector comprising a socket complementary to the plug and an external thread complementary to the internal thread,
the female connector being inserted into the annular gap of the male connector in a connected state,
the adhesive arrangement being provided in the annular gap and assigned an activation system, in such a way that activation of the adhesive arrangement takes place as a result of insertion of the female connector into the annular gap of the male connector.

2. The connecting system according to claim 1, wherein the activation system comprises encapsulation of the adhesive arrangement by at least one capsule.

3. The connecting system according to claim 2, wherein outer dimensions of the at least one capsule are matched to inner dimensions of the annular gap in such a way that the at least one capsule is receivable in the annular gap.

4. The connecting system according to claim 2, wherein the at least one capsule comprises a plurality of capsules, and wherein the adhesive arrangement comprises different adhesive components, which are accommodated in the plurality of capsules.

5. The connecting system according to claim 4, wherein the plurality of capsules with the different adhesive components are positioned in the annular gap in accordance with an activation sequence of the different adhesive components.

6. The connecting system according to claim 4, wherein the plurality of capsules are configured with such a low resistance that destruction of the plurality of capsules with release of the different adhesive components takes place during connection of the female connector to the male connector by contact between the female connector and the plurality of capsules.

7. The connecting system according to claim 6, wherein the plurality of capsules are configured in such a way that destruction of the plurality of capsules is perceptible to a user when the female connector is connected to the male connector.

8. The connecting system according to claim 2, wherein the at least one capsule is fixed in the annular gap with a force or form fit or materially.

9. The connecting system according to claim 1, wherein the activation system has a chemical or physical activation function assigned to the adhesive arrangement.

10. The connecting system according to claim 1, wherein the male connector and/or the female connector has a visually or haptically recognizable coding that allows a user to distinguish the male connector and/or the female connector from a known connecting system without an adhesive arrangement.
